(19) United States
(12) Patent Application Publication
(10) Pub. No.: US 2015/0352077 A1
(43) Pub. Date: Dec. 10, 2015

(54) Multi-Component Crystalline Particles for Inhalation Therapy

(71) Applicant: PROSONIX LIMITED, London (GB)

(72) Inventors: John Burns, Oxford (GB); Dipesh Parikh, Oxford (GB); Shyam Karki, Oxford, Oxfordshire (GB)

(73) Assignee: Prosonix Limited, London (GB)

(21) Appl. No.: 14/764,479

(22) PCT Filed: Jan. 29, 2014
(86) PCT No.: PCT/GB2014/050231

§ 371 Date: Jul. 29, 2015

(30) Foreign Application Priority Data
Jan. 31, 2013 (GB) 1301721.5

(51) Int. Cl. A61K 31/40; A61K 9/00; A61K 31/138; A61K 31/167; A61M 15/00; A61M 15/08

(52) U.S. Cl. CPC A61K 31/40; A61M 15/009; A61M 15/08; A61K 31/138; A61K 31/167; A61K 9/0078; A61K 9/0075; A61K 9/008; A61M 2202/064

(57) ABSTRACT

Pharmaceutical Preparations Multi-component crystalline particles and compositions, methods for their preparation, their uses in inhalation therapy and inhaler devices containing said particles are provided, in particular particles comprising glycopyrrolate. The particles can be prepared substantially free of excipients and agents other than active agents or their precursors in the presence of ultrasonic irradiation in a process comprising contacting a solution in a first flowing stream with an anti-solvent in a re-circulating second flowing stream, causing the mixing thereof and collecting crystals that are generated.

### FIELD OF THE INVENTION

The present invention relates to the preparation of multi-component crystalline particles of active agents suitable for use in inhalation therapy and for delivery by oral or nasal inhalation, wherein the particles can be prepared substantially free from excipients and other non-active agents. The present invention also provides particles and formulations prepared according to the process of the invention and their use in medicine.

### BACKGROUND OF THE INVENTION

The development of inhalation combination products raises the significant pharmaceutical challenge of maintaining a controllable ratio of drug components during various stages of drug formulation and drug delivery. Formulated products resulting from a physical mixture of active ingredients have been used to achieve the targeted material mix. However the co-deposition of actives derived from an aerosol cloud can lead to inconsistencies in the ratio of deposition in various regions of the lung, despite the use of a precise formulation mass ratio of the actives.

The use of multi-component particles (MCPs) can help to solve problems associated with combination products by helping to achieve consistent localised delivery. In addition, the use of multi-component particles eliminates the need for blending two micronized actives and therefore can help to avoid the possibility of localised high concentrations of highly potent active agents in the blend.

WO2002/28377A1 (Orion Corporation) claims crystalline spherical inhalation particles incorporating a combination of two or more different active ingredients and a process for their preparation whereby droplets containing active ingredients are suspended in a carrier gas and passed through a heated tube flow reactor for a predetermined residence time and temperature history and the particles produced collected. A combination particle of beclomethasone dipropionate and formoterol fumarate is exemplified.

WO2007/011989 (MAP Pharmaceuticals) claims inhalation particles where each discrete unagglomerated inhalation particle comprises two or more active pharmaceutical ingredients. WO2012/106575 (Novartis AG) claims dry powder formulations for inhalation comprising spray-dried particles and their use in the treatment of an obstructive or inflammatory airways disease. Each particle has a core of a first active ingredient in substantially crystalline form that is coated with a layer of a second active ingredient in substantially amorphous form that is dispersed in a pharmaceutically acceptable hydrophobic excipient.

WO2013/021199 (Prosonix Ltd) describes pharmaceutical compositions comprising a eutectic composition of two pharmaceutical ingredients for delivery to the lung. Microcrystalline particles of glycopyrronium bromide and salmeterol xinafoate, glycopyrronium bromide and indacaterol maleate and glycopyrronium bromide and formoterol fumarate prepared by UMAX processing (as defined in WO2010/007447) are exemplified.

Multi-component crystalline particles suitable for inhalation have proven difficult to prepare. Problems include significant variability in particle size, amorphous domains and unwanted polymorphs. This can affect the consistency of delivery to the lung and can lead to safety concerns, especially with multi-component particles containing highly potent P2 adrenergic receptor agonists. In addition, problems have been reported relating to the stability of particles containing muscarinic receptor antagonists. For example, WO2005/0105043 (Vectura Limited) indicates that glycopyrrolate has an acute problem with respect to its stability and the presence of non-crystalline or amorphous glycopyrronium bromide material can lead to significant physical instability.

Particle engineering techniques involving the use of ultrasound have been used to produce crystalline micro particles with a narrow size distribution. WO 2008/114052 (Prosonix Ltd) describes a process for preparing crystalline particles of one or more active principles in the presence of ultrasound. WO 2008/155570 (Prosonix Ltd) describes a process for preparing an emulsion or a dispersion comprising crystalline particles of at least one active principal by subjecting an emulsion or dispersion to ultrasonic irradiation.

### SUMMARY OF THE INVENTION

It has now been found that particularly advantageous multi-component particles can be prepared using ultrasound particle engineering techniques. It has now also been found that particular multi-component particles otherwise not amenable to combination particle engineering can be can be prepared using preferred ultrasound particle engineering techniques.

A particular advantage of multi-component particles and compositions according to the invention is the ability to achieve increased co-location of pharmacologically active ingredients. This approach has the potential to enhance the effect at the molecular and cellular level through synergistic pharmacological mechanisms, with the consequence of achieving acceptable efficacy at a reduced dose and an improved risk-benefit profile. Incorporation of active ingredients into a multi-component particle can also lead to a linked release of the active ingredients and therefore a more rapid onset of action of one or more actives. This effect has the potential to increase the likelihood of synergistic action of two or more actives with different dissolution rates. A faster rate of dissolution may lead to improvements in the onset of action and clinical efficacy.

In a first aspect, the invention provides multi-component crystalline particles for inhalation therapy comprising glycopyrrolate, including any pharmaceutically acceptable salts, esters, isomers or solvates thereof, wherein the particles can be prepared substantially free of excipients and agents other than active agents or their precursors and wherein the particles are prepared in the presence of ultrasonic irradiation in a process comprising contacting a solution in a first flowing stream with an anti-solvent in a re-circulating second flowing stream, causing the mixing thereof and collecting crystals that are generated.

A particular benefit of the invention is the ability to prepare multi-component particles comprising glycopyrrolate that are stable and crystalline. A further benefit provided by the present invention is the ability to prepare multi-component particles comprising glycopyrrolate which are substantially free from excipients and non-active components. The use of particles and compositions according to the invention could therefore help to reduce or to eliminate the deposition and build-up of excipients upon chronic repeat dosing of a patient. This could help to reduce associated systemic effects, for example the development of excipient intolerance or from the presence of surfactants leading to enhanced localised dissolution.

In one embodiment of the invention the glycopyrrolate is glycopyrronium bromide. In a further embodiment of the invention, the particles further comprise a long-acting β₂ adrenergic receptor agonist (LABA) or a pharmaceutically acceptable salt, ester, isomer, solvate or precursor thereof. In another embodiment the LABA is one or more of formoterol or salmeterol. In a further embodiment the particles comprise salmeterol xinafoate (SX) and glycopyrronium bromide (GB). In another embodiment the particles comprise formoterol fumarate (FF) and glycopyrronium bromide (GB). Glycopyrronium bromide can act as a solubilising agent for salmeterol xinafoate. The incorporation of SX and GB into a multi-component particle can therefore lead to an enhancement of the solubility of salmeterol.

In a further embodiment of the invention the particles further comprise a glucocorticosteroid or a pharmaceutically acceptable salt, ester, isomer, solvate or precursor thereof. In another embodiment, the particles comprise an additional LAMA or a pharmaceutically acceptable salt, ester, isomer, solvate or precursor thereof. In one embodiment of the invention the particles may comprise a eutectic composition. A eutectic composition may yield an increase in both equilibrium solubility and rate of dissolution of both pharmacologically active ingredients.

The selection of solvent and anti-solvent may be decided upon by the skilled person in accordance with the properties of the pharmacologically active ingredients utilised. In one embodiment of the invention, the anti-solvent is a dialkyl ether, such as tert-butyl methyl ether (TBME) or di-isopropyl ether (DIPE), and the solvent is an alcohol, such as methanol or ethanol. The use of a solvent and anti-solvent with restricted water content maybe important in producing multi-component particles comprising glycopyrrolate that are crystalline and of a suitable size for inhalation. In an embodiment of the invention, the solvent and anti-solvent contain less than 0.05% water.

The flow rate ratio of the anti-solvent:solution can be varied so as to achieve the formation of stable, crystalline particles according to the current invention with a suitable particle size distribution for inhalation. In one embodiment of the invention the flow rate ratio is greater than 20:1. In another embodiment, the flow rate ratio is greater than 700:1. In a further embodiment of the invention the flow rate ratio is greater than 1000:1. In another embodiment, the flow rate ratio is greater than 2500:1. In another embodiment, the flow rate ratio is greater than 5000:1. In a further embodiment the re-circulating anti-solvent velocity is greater than 0.5 m/s.

A further aspect of the invention provides a pharmaceutical composition deliverable from a pressurised metered dose inhaler, a dry powder inhaler, a nebulizer or a breath activated nasal inhaler comprising the multi-component particles of the invention. Methods of formulating compositions and pharmaceutically acceptable propellants, carriers and surface agents are known to one skilled in the art, for example by reference to texts such as Respiratory Drug Delivery: Essential Theory & Practice by Stephen Newman (Respiratory Drug Delivery Online, 2009). In one embodiment of the invention, the pharmaceutical composition deliverable from a pressurised metered dose inhaler is substantially free of excipients and or agents other than active agents or their precursors and a pharmaceutically acceptable propellant. In a further embodiment, the pharmaceutically acceptable propellant is selected from HFA134a or HFA 227.

In a further aspect of the invention, a dry powder inhaler, a pressurized metered-dose inhaler, a nebulizer or a breath activated nasal inhaler are provided incorporating a pharmaceutical composition according to the invention. Another aspect of the invention provides a method for treating a respiratory disease or disorder or a pulmonary disease or disorder in a patient using particles or compositions according to the invention. In one embodiment the disease is selected from asthma, chronic respiratory diseases, COPD and cystic fibrosis. A further aspect of the invention provides particles or compositions according to the invention for use in the treatment of a respiratory disease or disorder or a pulmonary disease or disorder. In one embodiment the disease is selected from asthma, chronic respiratory diseases, COPD and cystic fibrosis.

Another aspect of the invention provides a method of preparing multi-component crystalline particles according to the invention for inhalation therapy comprising glycopyrrolate, including any pharmaceutically acceptable salts, esters, isomers or solvates thereof, wherein the particles can be prepared substantially free of excipients and agents other than active agents or their precursors comprising contacting, in the presence of ultrasonic irradiation, a solution in a first flowing stream with an anti-solvent in a re-circulating second flowing stream, causing the mixing thereof and collecting crystals that are generated. A further aspect of the invention provides particles, compositions, inhalers and method of preparation and uses thereof substantially as described herein and with reference to the accompanying examples.

In one embodiment of the invention, the multi-component particles comprise glycopyrronium bromide and salmeterol xinafoate. In another embodiment of the invention, the multi-component particles comprise glycopyrronium bromide and formoterol fumarate. Other preferred multi-component crystalline particles prepared according to the present invention are glycopyrronium bromide combined with a glucocorticosteroid selected from the group fluticasone propionate, budesonide, mometasone, ciclesonide and beclomethasone.

### DETAILED DESCRIPTION OF THE INVENTION

The present invention provides multi-component particles which are prepared using particle engineering techniques. Multi-component particles of the invention comprise glycopyrrolate and at least one other pharmacologically active ingredient or precursor thereof.

Particles according to the current invention are crystalline and, when analysed by differential scanning calorimetry (DSC), show no significant exotherm which would indicate to the skilled person the presence of amorphous material. It will be appreciated that crystalline particles of the invention may comprise minor regions of amorphous material. By minor regions it is meant that the crystalline particles are less than 5% amorphous, preferably less than 1% amorphous.

Multi-component crystalline particles of the invention can be substantially free of excipients and agents other than active agents of their precursors. By substantially free it is meant that the crystalline particles contain less than 10% by weight of excipients and agents other than active agents of their precursors, preferably less than 5%, more preferably less than 2%. Multi-component particles of the current invention which are substantially free of excipients and agents other than active agents of their precursors may be treated with a surface agent after formation and before isolation, for example before isolation by spray drying.

The shape of the particles of the current invention are defined by the pharmacologically active ingredients and the process conditions employed. In one embodiment of the invention the particles are plate-shaped. In a further embodiment of the invention the particles are not spherical. In another embodiment of the invention the particles do not have substantially corrugated surfaces. Particles of the current invention have a size distribution suitable for oral or nasal inhalation, for example with a mass median aerodynamic diameter of up to 10 μm, up to 5 μm or up to 1 μm. The width of the particle size distribution may be quantified using the span which is a measure of the width of the distribution based on the 10%, 50% and 90% quantile. This value can be calculated using the formula (D90−D10)/D50. The span may be, for example less than 3, less than 2.5 or less than 2.

The pharmaceutical compositions of the current invention comprise glycopyrrolate. The word “glycopyrrolate” can be interchangeably used with “glycopyrronium”. Glycopyrrolate is a quaternary ammonium salt. Suitable counter ions are pharmaceutically acceptable counter ions including, for example, fluoride, chloride, bromide, iodide, nitrate, sulphate, phosphate, formate, acetate, trifluoroactetate, propionate, butyrate, lactate, citrate, tartrate, malate, maleate, succinate, benzoate, p-chlorobenzoate, diphenyl-acetate or triphenylacetate, o-hydroxyacetate, p-hydroxyacetate, 1-hydroxynapthalene-2-carboxylate, 3-hydroxynaphthalene-2-carboxylate, methanesulfonate and benzenesulfonate. A preferred counter ion of glycopyrrolate is bromide. The bromide salt of glycopyrrolate is glycopyrronium bromide, which is chemically known as 3-(2-cyclopentyl-2-hydroxy-2-phenylacetoxy)-1,1-dimethylpyrrolidinium bromide. Glycopyrrolate has two chiral centres and can exist in four stereoisomeric forms. Compositions of the current invention may comprise racemic glycopyrrolate, one of the enantiomers, one of the diastereomers or a mixture thereof.

In addition, particles of the current invention may comprise active agents selected from β₂ adrenergic receptor agonists, anti-cholinergics including muscarinic antagonists and glucocorticosteroids. Long-acting β₂ adrenergic receptor agonists (LABAs) and long-acting muscarinic antagonists (LAMAs) have a prolonged duration of action, such as greater than 12 hours, and are therefore suitable for once- or twice-daily dosing.

Preferred β₂ adrenergic receptor agonists are LABAs, preferably selected from the group consisting of formoterol, salmeterol, carmoterol, indacaterol, vilanterol, arformoterol, bambuterol, isoproterenol, milveterol, clenbuterol, olodaterol and salts, esters, polymorphs, hydrates, solvates or isomers thereof. A particularly preferred salt of formoterol is formoterol fumarate (FF). A particularly preferred salt of salmeterol is salmeterol xinafoate (SX). β₂ agonists may also be short acting β₂ agonists such as fenoterol, salbutamol, levalbuterol, procaterol, terbutaline, pirbuterol, procaterol, metaproterenol, bitolterol, ritodrine, albuterol and salts, esters, polymorphs, hydrates, solvates or isomers thereof, preferably fenoterol hydrobromide. Formoterol fumarate of the current invention may be in an anhydrous form or present as a hydrate, for example as a monohydrate or dihydrate. Compositions of the current invention may comprise racemic formoterol, one of the enantiomers, one of the diastereomers or a mixture thereof.

Preferred anti-cholinergics are LAMAs preferably selected from the group consisting of tiotropium, aclidinium, darotropium, umedlidinium and salts, esters, polymorphs, hydrates, solvates or isomers thereof. A preferred short-acting muscarinic antagonist is ipratropium and salts, esters, polymorphs, hydrates or solvates thereof. Particularly preferred muscarinic antagonists are selected from the group consisting of tiotropium bromide, ipratropium bromide, aclidinium bromide, darotropium bromide or umeclidinium bromide and salts, esters, polymorphs, hydrates, solvates or isomers thereof.

Preferred glucocorticosteroids are selected from the group consisting of mometasone, beclamethasone, budesonide, fluticasone, ciclesonide or triamcinolone and salts, esters, polymorphs, hydrates, solvates or isomers thereof, preferably beclamethasone dipropionate, fluticasone propionate, fluticasone furoate, mometasone furoate, or budesonide. Preferred combinations of glycopyrrolate and LABA are glycopyrronium bromide and salmeterol xinafoate (GB/SX) and glycopyrronium bromide and formoterol fumarate (GB/FF). Preferred combinations of glycopyrrolate and an additional LAMA are glycopyrronium bromide and tiotropium bromide and glycopyrronium bromide and aclidinium bromide.

The multi-component particles may have a molar ratio of 100:1 to 1:1, 50:1 to 1:1, 10:1 to 1:1, 9:1 to 1:1, 4:1 to 1:1 or 2:1 to 1:1. Alternatively, the multi-component particles may have a mass ratio of 100:1 to 1:1, 50:1 to 1:1, 10:1 to 1:1, 9:1 to 1:1, 4:1 to 1:1, 2:1 to 1:1. Preferred particles of the current invention have a glycopyrrolate to LABA molar ratio of 8:1, 7.5:1, 2:1 or 1:1, or a glycopyrrolate to LABA mass ratio of 8:1, 7.5:1, 2:1 or 1:1. For example GB:FF with a mass ratio of 7.5:1 or 2:1, GB:FF with a molar ratio of 1:1, GB:SX with a mass ratio of 7.5:1 or 2:1 or GB:SX with a molar ratio of 1:1.

Multi-component particles of the current invention may comprise a eutectic composition. In another aspect of the invention the multi-component particles do not comprise a eutectic composition. A eutectic composition has a lower melting point than that of either pure compound. A eutectic composition is clearly differentiated from the phenomenon of co-crystal formation. A person skilled in the art will appreciate that in a eutectic composition the two constituent materials are independently crystalline whereas in the case of a co-crystal a completely new crystalline phase forms and in effect replaces the separate crystalline phases with respect to the component molecules within each unit cell. Eutectic compositions can have advantages related to the reduced thermodynamic stability of the composition leading to an increase in both equilibrium solubility and rate of dissolution of both pharmacologically active ingredients. In order to determine whether or not a eutectic composition exists or can be found, a person skilled in the art could use differential scanning calorimetry (DCS) to verify the melting point and the magnitude of melting point suppression. The particles comprising a eutectic composition may further comprise an excess of at least one of the pharmacologically active ingredients.

The particles of the invention can be prepared using equipment as described in WO 2008/114052 and other systems employed by the person skilled in the art. The particles are prepared in the presence of ultrasonic irradiation in a process comprising contacting a solution in a first flowing stream with an anti-solvent in a re-circulating second flowing stream, causing the mixing thereof, and collecting crystalline particles that are generated.

The first flowing stream and the second flowing stream are contacted in the presence of ultrasonic irradiation, for example in an ultrasonic flow cell. The ultrasound induces nucleation and so crystallisation.

The solvent, used to form the solution, and the anti-solvent should be selected as suitable for a particular combination of pharmacologically active ingredients. Preferably the solvent is an alcohol, for example methanol or ethanol. The anti-solvent should be an organic solvent. Preferably, the anti-solvent is a non-polar solvent, such as a non-polar aprotic solvent. More preferably the anti-solvent is a di-alkyl ether, for example tert-butyl methyl ether (TBME) or di-isopropyl ether (DIPE). Without being bound by theory, the selection of a dialkyl ether solvent, for specific combinations of active ingredients, may assist in the formation of multi-component particles rather than the formation of particles of individual active ingredients. It will be appreciated that the solution may also comprise an additional co-solvent and that the anti-solvent may comprise more than one anti-solvent. The ratio of the volume of solution to volume of non-solvent is typically between 1:5 to 1:40, preferably between 1:15 to 1:30, for example 1:20 or 1:24.

The amount of water in the solvent and anti-solvent may be an important parameter in the production of stable multi-component crystalline particles comprising glycopyrrolate of a suitable size for inhalation therapy. The water content, of the solvent and anti-solvent, as measured for example by a Karl Fischer titration, should be preferably less than 0.1% w/w, preferably less than 0.075% w/w, such as less than 0.05% w/w.

It will be appreciated that the temperature of the solution and anti-solvent should be selected in accordance with the substances to be crystallised. The solution and anti-solvent may be held at the same temperature. Alternatively the solution may be at a higher or lower temperature than the anti-solvent. Typically, the temperature of the anti-solvent may lie between −10° C. and 60° C., such as between 0° C.-20° C. or between 5° C.-10° C. Ultrasound irradiation is employed at a power density appropriate for the formation of crystals of the desired size. Typically, the ultrasound power density is 10-100 W/L, preferably from 25-75 W/L.

The flow rate ratio of the anti-solvent:solution can be varied so as to achieve the formation of stable, crystalline particles according to the current invention with a suitable particle size distribution for inhalation. Suitable flow rate ratios of anti-solvent to solution are greater than 20:1, greater than 700:1, greater than 1000:1, greater than 2500:1 or greater than 5000:1. An example of the flow rate ratio (5260:1) used in the present invention is to be found in the examples wherein the anti-solvent is re-circulated at a flow rate of 2.63 L/min and the solution is added at 0.5 mL/min.

The velocity of the re-circulating anti-solvent stream and the solution addition rate are important factors in producing multi-component crystalline particles of the current invention.

For example, when the solution and the anti-solvent stream are contacted in an ultrasonic flow cell, at the point of entering the ultrasonic flow cell the velocity should be greater than 10 cm/s, preferable greater than 0.5 m/s, more preferably greater than 1 m/s, up to about 10 m/s.

Multi-component crystalline particles prepared by the current invention may be harvested and isolated using conventional means, for example by filtration or by spray-drying.

Isolated multi-component particles may be further treated to reduce the amount of any residual solvent or anti-solvent and/or to form a more hydrated material with the potential for greater long term stability. Particles may be subjected to further drying, for example under vacuum. In addition, or as an alternative, particles may be subjected to a humid environment, for example placed within a humidity chamber. The relative humidity at the temperature of the process may be greater than 30%, for example greater than 40%, such as between 40 and 70%, or between 45 and 55%. The process may be carried out at ambient temperature or an elevated temperature, for example at a temperature greater than around 16° C., such as a temperature in the range 16° C.-40° C. or 18-25° C.

The process may be monitored by analysis of the solvent/anti-solvent and water content of the particles. The process may be stopped at the point whereby the residual solvent/anti-solvent are at acceptable levels. For example the particles may be subjected to the humid environment for a period of time greater than 12 hours, such as between 18 and 30 hours or between 22 and 28 hours.

The pharmaceutical compositions of the present invention can be administered by a dry powder inhaler, a pressurised metered dose inhaler, a nebulizer or a breath activated nasal inhaler. The invention therefore provides a dry powder inhaler, a pressurized metered-dose inhaler, a nebulizer or a breath activated nasal inhaler comprising a composition of the invention.

A pharmaceutical composition of the present invention that is deliverable from a pressurised metered dose inhaler may be substantially free of excipients and or agents other than active agents or their precursors and a pharmaceutically acceptable propellant. By substantially free it is meant that the composition contain less than 10% by weight of excipients and agents other than active agents or their precursors and a pharmaceutically acceptable propellant, preferably less than 5% by weight, such as less than 2.5%. Suitable propellants may be selected from the group of HFA propellants, for example HFA134a (1,1,1,2-tetrafluoroethane) or HFA 227 (1,1,1,2,3,3,3,-heptafluoropropane).

### BRIEF DESCRIPTION OF DRAWINGS

FIG. 1 shows the DSC profile of particles isolated by filtration from Example 1.

FIG. 2 shows the DSC profile of particles isolated by spray drying from Example 1.

FIG. 3 shows the results of HPLC analysis of the particles from Example 1.

FIG. 4 shows the DSC profile of particles isolated by filtration from Example 2.

FIG. 5 shows the DSC profile of particles isolated by spray drying from Example 2.

FIG. 6 shows the results of HPLC analysis of the particles from Example 2.

FIG. 7 shows the results of stability testing (GB/SX GB-SX 2:1 mass MCP).

FIG. 8 shows the results of stability testing (GB-FF 2:1 Mass MCP).

FIG. 9 shows the results of stability testing (GB-SX 1:1 Molar MCP).

FIG. 10 shows the results of stability testing (GB-FF 1:1 Molar MCP).

FIG. 11 shows a schematic of the method of quantification of co-location performance.

FIG. 12 shows an analysis of active ingredient co-location.

FIG. 13 shows the dissolution of salmeterol delivered from salmeterol xinafoate-glycopyrronium bromide combination inhalers (n=3; mean).

FIG. 14 shows the dissolution of glycopyrronium bromide delivered from salmeterol xinafoate-glycopyrronium bromide combination inhalers (n=3; mean).

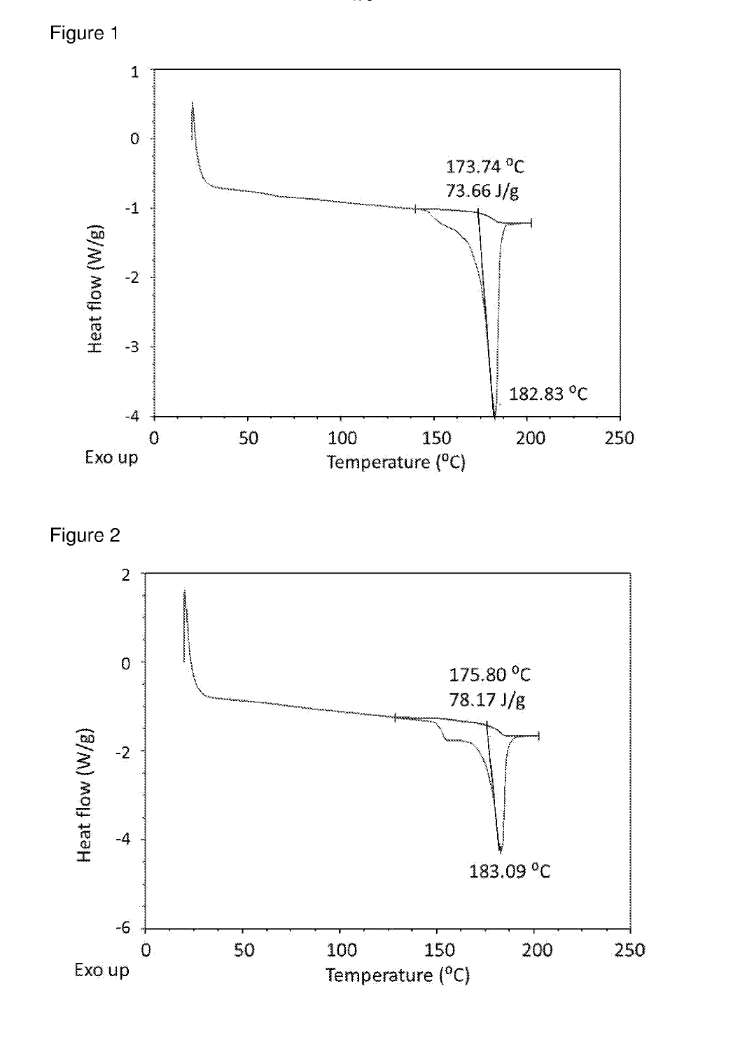

The invention will now be described in more detail with reference to and by way of examples which are intended to be illustrative only. It is to be understood that the examples and figures are not to be construed as limiting the scope of the invention in any way.

### EXAMPLES

### Example 1

### Glycopyrronium Bromide (GB) and Formoterol Fumarate (FF)

Methanolic solutions of GB/FF were prepared and added to re-circulating TBME at room temperature at an addition rate of 0.5 ml/min, solution /non-solvent 1/20, using 40 w ultrasound power using a thick probe based system. Immediate recrystallisation and formation of uniform slurry was observed in all cases. Material isolated by filtration was crystalline as indicated by differential scanning calorimetry (DSC).

For GB:FF (7.5:1) in MeOH/TBME, experiment parameters were as follows.
Solution concentration: 25% (6.8 g in 27 ml methanol)
Volume TBME: 648 ml
Solution-non-solvent ratio: 1/24 V/V
Reaction vessel temperature: 7.4+/−0.2° C.
Solution addition rate: 0.5 ml/min
Solution addition velocity: 0.042 m/s
Solution addition tube diameter: 0.5 mm
Duration of addition: 60 mins
Re-circulation rate: 0.9 L/min
Velocity of re-circulating anti-solvent stream: 1.4 m/s
Flow rate ratio: 5260:1
Ultrasound: 40 W
Moisture content in the processed slurry (by Karl Fischer titration): 0.045%

### Isolation by Filtration

Immediate re-crystallization was observed resulting in the production of fine plate shaped particles. The particle size was acceptable for inhalation formulation (d50-1.86 uM). Differential scanning calorimetry (DSC) indicated crystalline nature of the final material (endotherm peak 183° C.).

Particle size distribution (μM)×10=0.67, ×50=1.86, ×90=4.49. Span=2.05

### Isolation by Spray Drying

Spray drying was performed in an open loop system with outlet T: 53-55 degrees C., Inlet T: 80 degrees C., 100% aspirator, N₂ to nozzle: 24 LPM, N₂ gas flow rate: 316 LPM, nozzle size 0.5 mm and slurry flow rate: 10 ml/min. Differential scanning calorimetry (DSC) indicated crystalline nature of the final material (endotherm peak 183° C.). Particle size is in the acceptable range for inhalation d50=1.78 μM, smaller than that of pressure filtered material.

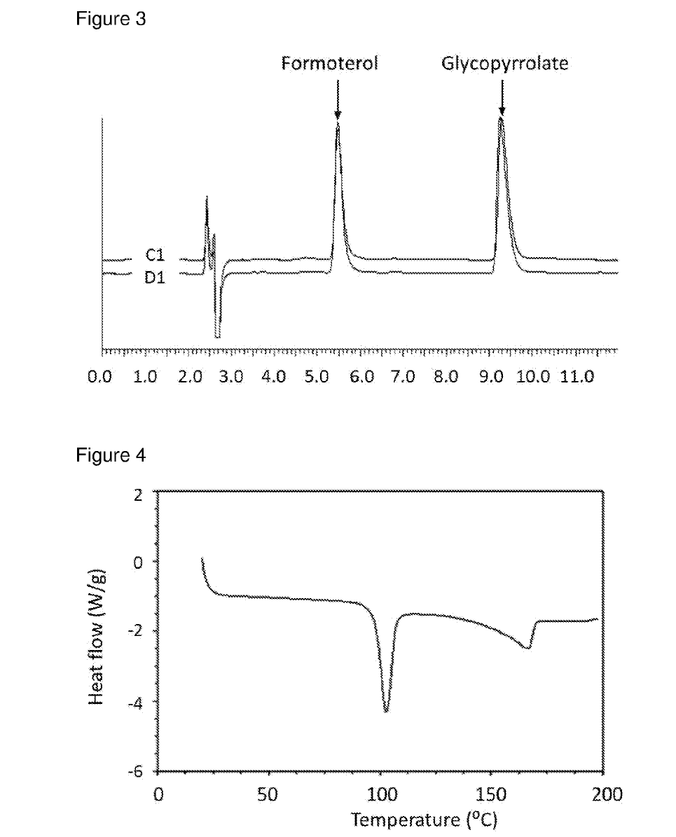

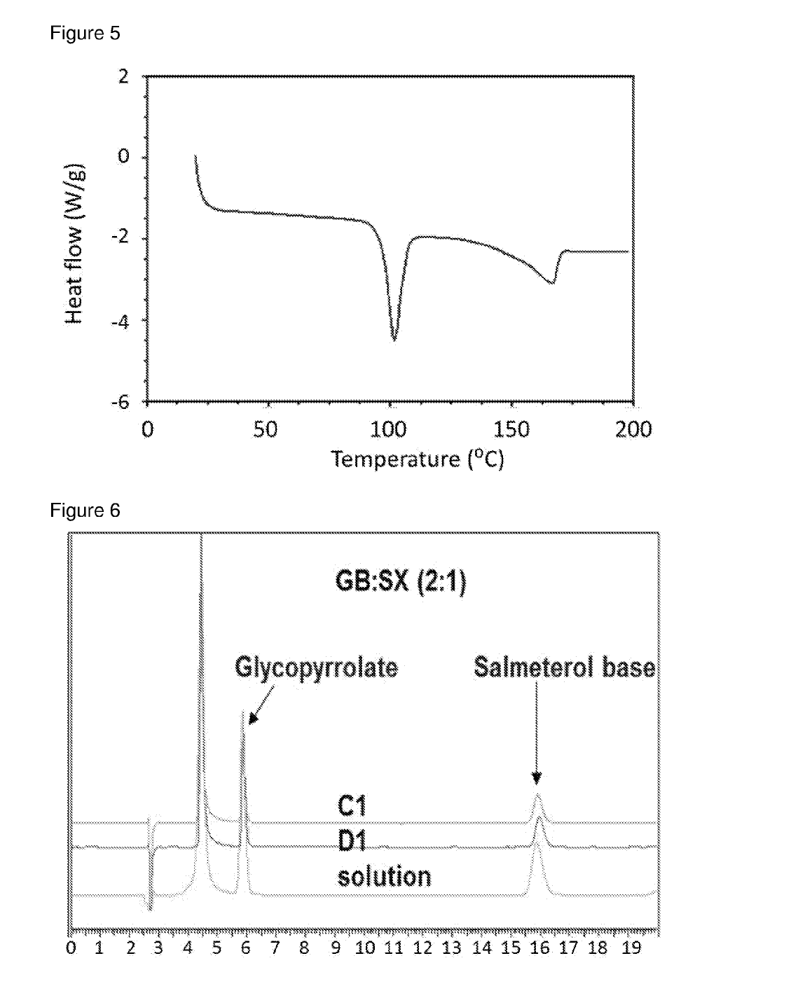

Particle size distribution (μM)×10=0.68, ×50=1.78, ×90=3.98. Span=1.85
FIG. 3 shows the result of HPLC analysis.
Ratio of active pharmaceutical ingredient (API):
   Filtered sample PXLB053-104-C1: 8.2:1 (GB:FF)
   Spray dried sample PXLB053-104-D1: 8.1:1 (GB:FF)
The GB/FF ratio was maintained during processing using ultrasonic particle engineering.

### Summary

As shown above combination particles were successfully prepared. The particles were of suitable size for inhalation d50<2 μm. The final material exhibited highly crystalline behaviour. Moisture content analysis by KF (Karl Fischer) and TGA (Thermo Gravimetric Analysis) confirmed that the final material contained di-hydrated form of Formoterol fumarate. Material isolated by spray drying was free flowing, less electrostatic and exhibited low bulk density compared with pressure filtered material. The ratio of GB and FF was retained at a pharmaceutical acceptable standard.

### Example 2

### Glycopyrronium Bromide (GB) and Salmeterol Xinafoate (SX)

Methanolic solutions of GB/SX were prepared in different ratios (4:1, 2:1, and 1:1) and added to re-circulating DIPE at room temperature at an addition rate of 0.5 ml/min, solution/non-solvent 1/20 using 40 W US power using a thick probe based system. Immediate recrystallisation and formation of uniform slurry was observed in all cases. Material isolated by filtration was crystalline as indicated by DSCs.
For GB:SX (2:1) in MeOH/DIPE, experiment parameters were as follows.
Solution concentration: 25% (6.8 g in 27 ml methanol)
Volume DIPE: 648 ml
Solution-non-solvent ratio: 1/24 VN
Reaction vessel temperature: 7.4+/−0.2° C.
Solution addition rate: 0.5 ml/min
Solution addition velocity: 0.042 m/s
Solution addition tube diameter: 0.5 mm
Duration of addition: 60 mins
Re-circulation rate: 2.63 L/min
Velocity of re-circulating anti-solvent stream: 0.9 m/s
Flow rate ratio: 5260:1
Ultrasound: 40 W
Moisture content in the processed slurry (by Karl Fischer titration): 0.015%

Isolation by Filtration

Immediate re-crystallization was observed resulting in the production of fine plate shaped particles (d50>2.0 μm, due to agglomeration). Differential scanning calorimetry (DSC) indicated crystalline nature of the final material.
Particle size distribution (uM)×10=0.61, ×50=2.04, ×90=6.11. Span=2.69

### Isolation by Spray Drying

Spray drying was performed in an open loop system with outlet T: 70-73 degrees C., 100% aspirator, N₂ to nozzle: 24 LPM, N₂ gas flow rate: 316 LPM, nozzle size 0.5 mm and slurry flow rate: 10 ml/min. Differential scanning calorimetry (DSC) indicated crystalline nature of the final material. Particle size is in the acceptable range for inhalation d50=1.78, smaller than that of pressure filtered material.

Particle size distribution (μM)×10=0.60, ×50=1.78, ×90=4.73. Span=2.32
FIG. 6 shows the result of HPLC analysis
Ratio: GB:SX
Solution=2.09
C1 (isolation by filtration)=2.10
D1 (isolation by spray drying)=2.10
The GB/SX ratio was maintained during processing using ultrasonic particle engineering.

### Summary

Combination particles of GB/SX (2:1 W/VV) were successfully prepared by ultrasonic techniques using a MeOH/DIPE system. The particles were of suitable size for inhalation. The final material exhibited highly crystalline behaviour. Material isolated by spray drying was free flowing, less electrostatic and exhibited low bulk density compared to pressure filtered material. The ratio of GB and SX was retained at a pharmaceutical acceptable standard.

### Stability Analysis

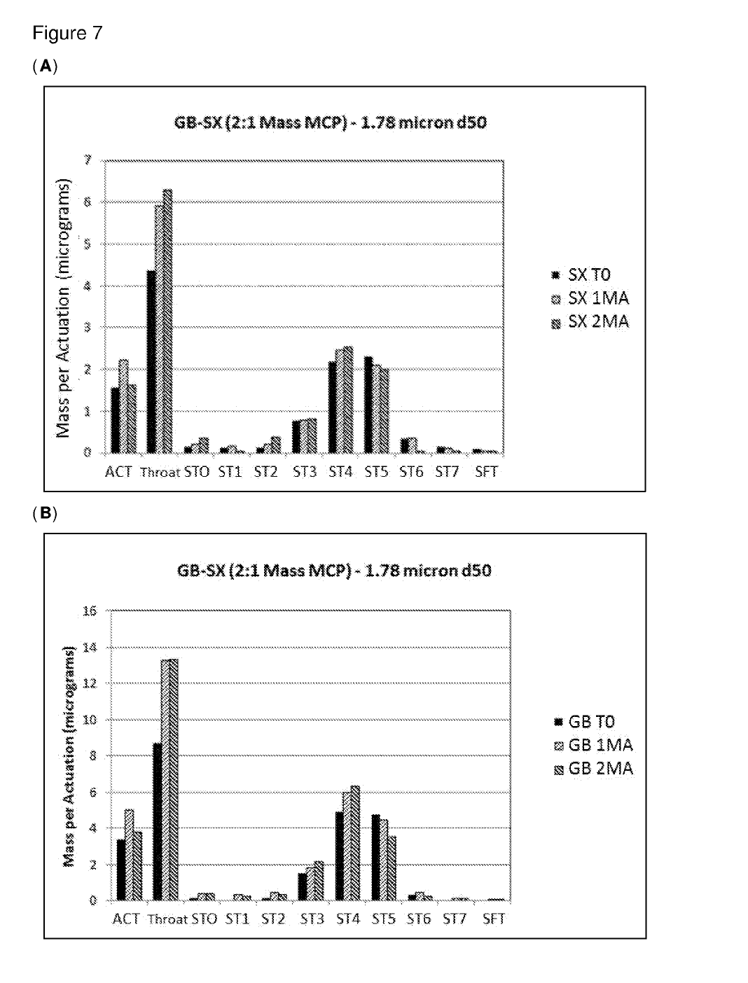

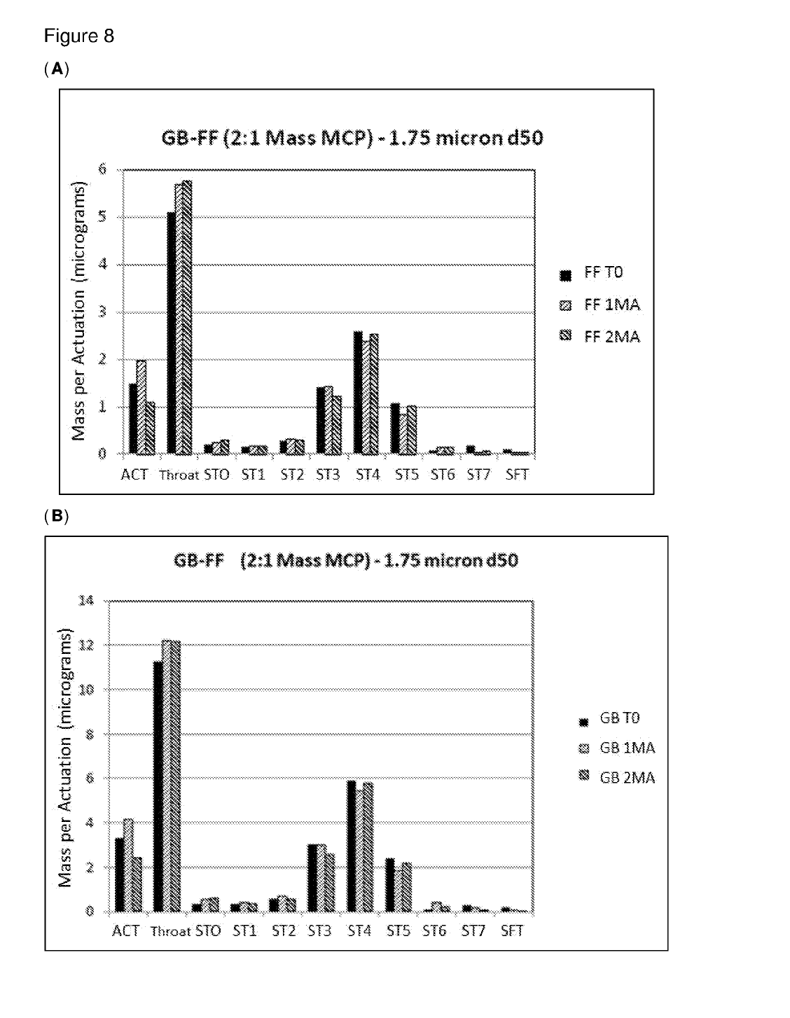

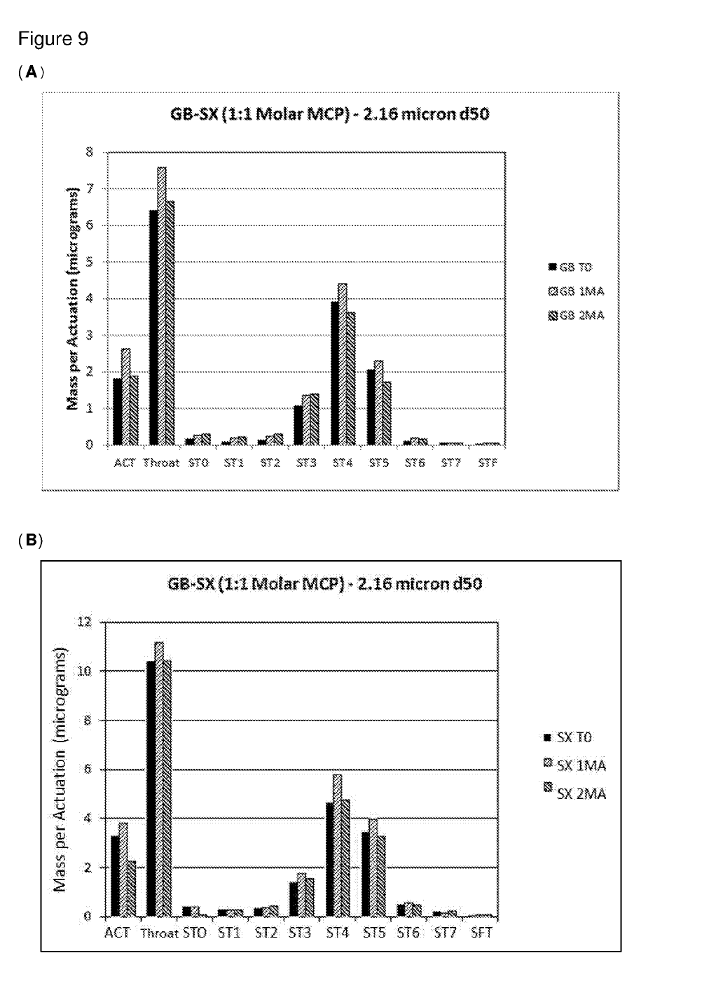

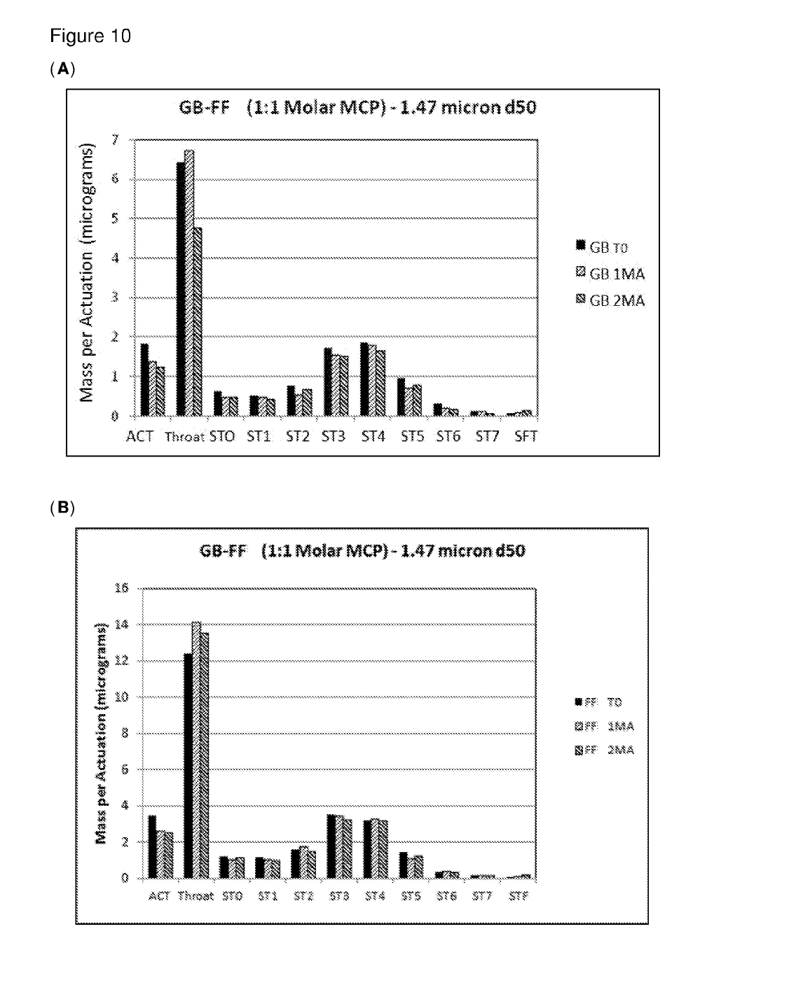

Stability analysis was performed under accelerated conditions (75% relative humidity, 40° C.) using coated cans and without overwrapping. The following formulations were tested:
Multi-component GB:SX particles at 1:1 molar ratio
Multi-component GB:FF particles at 1:1 molar ratio
Multi-component GB:SX particles at 2:1 mass ratio
Multi-component GB:FF particles at 2:1 mass ratio
   Formulations were analysed after 1 and 2 months using an Anderson Cascade Impactor (ACI). Results are shown in FIGS. 7 to 10.

### Co-Location Analysis

The data from the ACI stability testing was further analysed to investigate the co-location of the active ingredients compared to those of (un-optimised) blends of active pharmaceutical ingredients (APIs). The ACI traces were converted to represent the proportion of API at each stage compared to the total delivered.

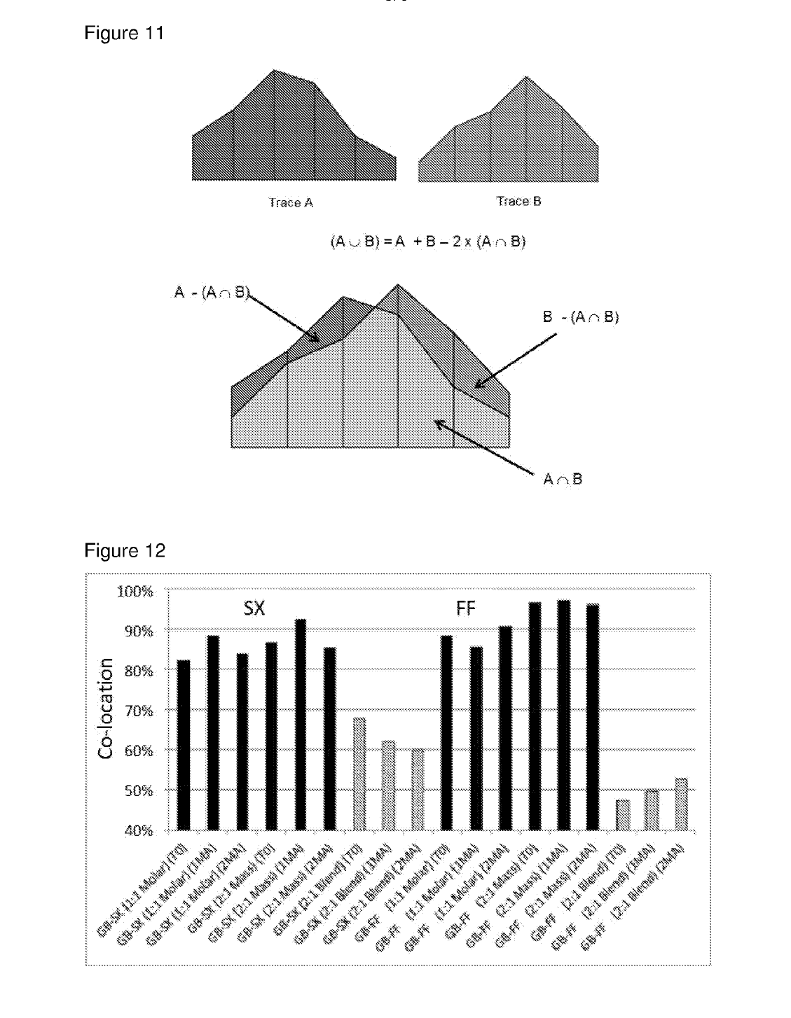

A single number representation of how well two traces match can be calculated as the ratio of the area of intersection of the two traces divided by the area of the union of the two traces (see FIG. 11). For identical traces this will take a value of 1, and for traces with no overlap it will take the value of zero. Mathematically it is represented as,

Co-Location Performance (%)=Area of ((A∩B)/Area of (A∪B))*100

The results are given in FIG. 12.

### Dissolution Rate Analysis

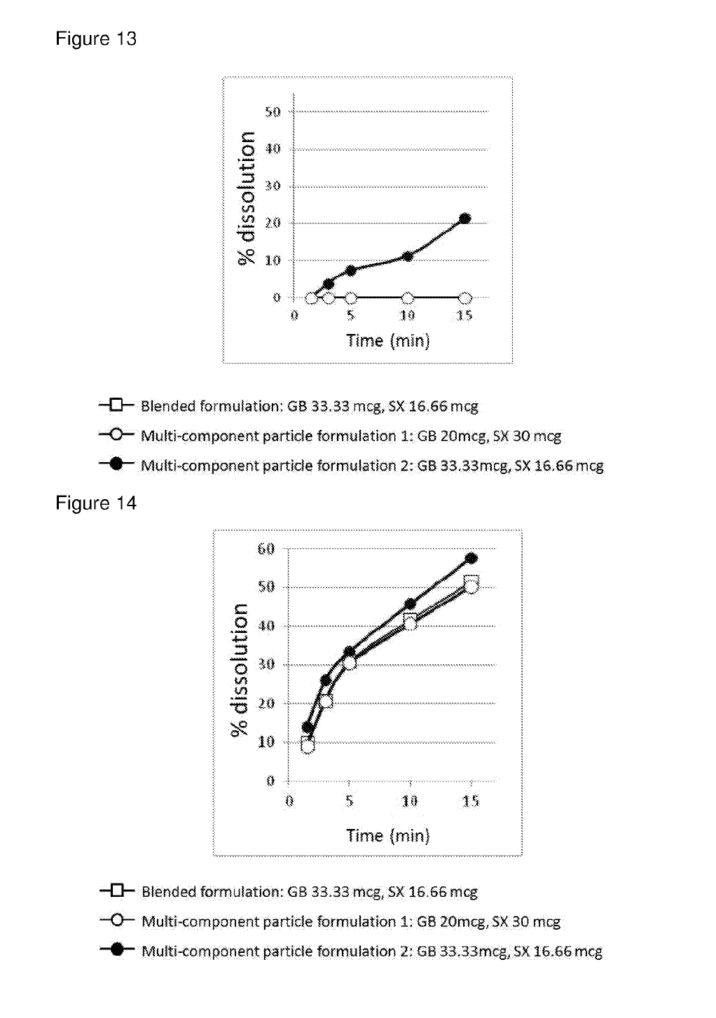

The dissolution of active ingredients was measured after delivery of ten actuations of each combination pressurised metered dose inhaler into a Twin Stage Impinger and collection of particles on the wetted surface of a Transwell inserted at stage 2. The dissolution rate in water for salmeterol from either a blended formulation or multi component particle is shown in FIG. 13. The dissolution rate in water for glycopyrronium bromide from either a blended formulation or multi component particle is shown in FIG. 14. This data indicates a significantly higher dissolution of salmeterol from the multi component particle formulation (GB 20 mcg, SX 30 mcg) when compared to either a blended formulation (GB 33.33 mcg, SX 16.66 mcg) or the multi component particle formulation (GB 33.33 mcg, SX 16.66 mcg). No significant differences in glycopyrronium bromide dissolution rate were observed between the formulations tested.

Solubility testing was also carried out in 10, 50 and 75% methanol which indicated no significant difference in glycopyrronium dissolution between MCPs and a blended formulation. Salmeterol dissolution from the blended composition was enhanced with increasing methanol concentrations, showing comparable dissolution to the GB 20 mcg/SX 30 mcg MCP formulation at 10 and 50% methanol and greater dissolution at 75% methanol.

### Residual Anti-Solvent Reduction/Hydration Process Step

General Procedure

Multi-component particles isolated from the described process were vacuum dried and then analysed for TMBE and water content. The particles were placed into a humidity chamber with a relative humidity of 50±2% and at a temperature of 20±2° C. for a period of 24 hours. The particles were re-analysed for TMBE and water content. The results are shown in the tables below:

GB-FF (4:1, wt.) vacuum dried material (0.5-1 g)

| | TBME (%) | Water (%) |
|---|---|---|
| Vacuum dried material | 0.92 | 0.84 |
| Hydrated material | 0.20 | 1.79 |

GB-FF (1:1, Molar) vacuum dried material (50-100 mg)

| | TBME (%) | Water (%) |
|---|---|---|
| Vacuum dried material | 3.14 | 1.33 |
| Hydrated material | 0.98 | 4.63 |

No significant change in particle size distribution was observed in either case.

What is claimed:

1. Multi-component crystalline particles for inhalation therapy comprising glycopyrrolate and a long-acting β₂ adrenergic receptor agonist (LABA), including any pharmaceutically acceptable salts, esters, isomers or solvates thereof, wherein the particles are substantially free of excipients and agents other than active agents and wherein the particles are prepared in the presence of ultrasonic irradiation in a process comprising contacting a solution in a first flowing stream with an anti-solvent in a re-circulating second flowing stream, causing the mixing thereof and collecting crystals that are generated.

2. Particles according to claim 1 wherein the glycopyrrolate is glycopyrronium bromide (GB).

3. (canceled)

4. Particles according to claim 1 wherein the LABA is one or more of formoterol or salmeterol.

5. Particles according to claim 1 comprising salmeterol xinafoate (SX) and glycopyrronium bromide (GB).

6. Particles according to claim 1 comprising formoterol fumarate (FF) and glycopyrronium bromide (GB).

7.-8. (canceled)

9. Particles according to claim 1 comprising a eutectic composition.

10. Particles according to claim 1 whereby the anti-solvent is a dialkyl ether, such as tert-butyl methyl ether or di-isopropyl ether, and the solvent is an alcohol, such as methanol or ethanol.

11. Particles according to claim 1 whereby the solvent and anti-solvent contain less than 0.05% water.

12.-14. (canceled)

15. Particles according to claim 1 wherein the flow rate ratio of the anti-solvent:solution is greater than 5000:1.

16. Particles according claim 1 whereby the re-circulating anti-solvent velocity is greater than 0.5 m/s.

17. A pharmaceutical composition deliverable from a pressurised metered dose inhaler, a dry powder inhaler, a nebulizer or a breath activated nasal inhaler comprising particles according to claim 1.

18. A pharmaceutical composition deliverable from a pressurised metered dose inhaler according to claim 17 which is substantially free of excipients and or agents other than active agents and a pharmaceutically acceptable propellant.

19.-24. (canceled)

25. A method of preparing multi-component crystalline particles for inhalation therapy comprising glycopyrrolate and a long-acting β₂ adrenergic receptor agonist (LABA), including any pharmaceutically acceptable salts, esters, isomers or solvates thereof, wherein the particles can be prepared substantially free of excipients and agents other than active agents, the method comprising contacting. In the presence of ultrasound irradiation, a solution in a first flowing stream with an anti-solvent in a re-circulating second flowing stream, causing the mixing thereof and collecting crystals that are generated.

26. (canceled)

27. A method according to claim 25 wherein the LABA is one or more of formoterol or salmeterol.

28. A method according to claim 25 wherein the particles comprise salmeterol xinafoate (SX) and glycopyrronium bromide (GB).

29. A method according to claim 25 wherein the particles comprise formoterol fumarate (FF) and glycopyrronium bromide (GB).

30. A method according to claim 25 whereby the anti-solvent is a dialkyl ether, such as tert-butyl methyl ether or di-isopropyl ether, and the solvent is an alcohol, such as methanol or ethanol.

31. A method according to claim 25 whereby the solvent and anti-solvent contain less than 0.05% water.

32. A method according to claim 25 wherein the flow rate ratio of the anti-solvent:solution is greater than 5000:1.

33. A method according to claim 25 wherein the re-circulating anti-solvent velocity is greater than 0.5 m/s.
